(19) United States
(12) Patent Application Publication
(10) Pub. No.: US 2014/0025033 A1
(43) Pub. Date: Jan. 23, 2014

(54) Non-Invasive Fat Reduction by Hyperthermic Treatment

(71) Applicants: Mirko G. Mirkov, Chelmsford, MA (US); Bo Chen, Burlington, MA (US)

(72) Inventors: Mirko G. Mirkov, Chelmsford, MA (US); Bo Chen, Burlington, MA (US)

(73) Assignee: CYNOSURE, INC., Westford, MA (US)

(21) Appl. No.: 13/991,289

(22) PCT Filed: Dec. 2, 2011
(86) PCT No.: PCT/US11/63113

§ 371 Date: Oct. 3, 2013

Related U.S. Application Data
(60) Provisional application No. 61/419,440, filed on Dec. 3, 2010.

(51) Int. Cl. A61B 18/20; A61N 5/06

(52) U.S. Cl. CPC A61B 18/20; A61N 5/062
USPC 604/501; 606/13; 606/3

(57) ABSTRACT

The present disclosure relates systems and methods for tissue remodeling, that ameliorate fat deposits by disrupting adipocytes through low-temperature extended treatment time approaches, in conjunction with selective treatment and/or localized cooling of the treatment site to prevent or minimize damage to non-target tissues.

### REFERENCE TO RELATED APPLICATIONS

This application claims priority to and the benefit of U.S. Provisional Application No. 61/419,440, filed on Dec. 3, 2010, the entire contents of which are incorporated by reference.

### FIELD OF THE INVENTION

The present disclosure relates to the field of aesthetic medical procedures. Specifically, the disclosure provides for systems and methods of tissue remodeling by ameliorating fat deposits.

### BACKGROUND

Eliminating unwanted body fat has become important from both health and aesthetic standpoints. Reducing these unwanted fat deposits (e.g., “love handles”) in various anatomic locations such as the flanks, abdomen, and thighs has been shown to improve overall health, with positive effects on one's self image. Routines such as dieting and exercise can reduce body fat, but certain areas of the body may not be responsive to such measures, and reductions in fat accumulation can be difficult to achieve without surgical intervention and physical removal. Liposuction is a reasonable therapeutic option for this condition. Although dramatic clinical improvement can be achieved with this surgical procedure, there is considerable associated postoperative recovery and monetary expense. As such, noninvasive or minimal invasive procedures with quick postoperative recovery and a low side-effect profile are in considerable demand. Various methods for localized fat destruction are emerging as alternatives to traditional liposuction. Non-invasively achieved fat reduction has been developed using lasers, focused ultrasound, radiofrequency devices, and selective cryolysis. Removal of fat from irradiation of adipocytes with a 635 nm wavelength laser has been claimed, but further evidence including histological studies is still needed to further establish this approach. Focused ultrasound and radiofrequency devices rely on acute heating and therefore thermally damaging deep fat in a localized area, but deep nodules and prolonged pain are often reported as side effects.

### SUMMARY OF THE INVENTION

The invention disclosed herein relates to devices and methods for low-temperature treatments that disrupt subcutaneous adipose tissues. These treatments are suitable for tissue remodeling and cosmetic applications. The invention contemplates achieving a balance between heat deposition and cooling, such that an optimal temperature range in the treatment site is maintained. Specifically, the invention provides for a tissue treatment method including delivering to a treatment site within a tissue of a patient sufficient energy to heat the tissue to a mean temperature above 40° C.; and maintaining a temperature below 47° C. within and proximal to the treatment site, thereby damaging adipocytes within the treatment site without substantial damage to epithelial or vascular tissues proximal to the treatment site. Heating of tissues within the treatment site is accomplished with laser radiation having a wavelength capable of deep tissue penetrance, such as in the near infrared spectra, e.g., ranging from about 800 nm to about 1200 nm, for example but not limited to a 1064 nm laser. Treatment times can range from about 2 to about 60 minutes, and depend on the particular fluence value. Accordingly, a useful power density range for such treatments includes an average power density of about 1-10W/cm2, and preferably an average power density of about 4-6W/cm2.

Thermal control of the treatment site is achieved with a number of approaches, that can be employed individually and in combination. In one embodiment, energy is delivered to the treatment site in the form of periodic pulsed radiation. In one embodiment, the step of maintaining a temperature below 47° C. within and proximal to the treatment site is effected at least in part by determining the temperature as a function of time of the treatment site, and modulating the delivery of energy from the energy source in response thereto. The temperature determinations can be effected by, for example, thermal imaging sensors. In some embodiments, the step of maintaining a temperature below 47° C. within and proximal to the treatment site is effected at least in part by modulating the delivery of energy from the energy source. Some useful ways of controlling temperature occur through such approaches as application of an external cooling means, such as a contact chiller, or through convection cooling based on exposing the treatment site to one or more streams of relatively cool air. Cooling may occur simultaneously with treatment, and can extend beyond the end of treatment for an appropriate time, to reduce post-operative inflammation and pain. Cooling can be intermittent during energy delivery as well, for example the cooling systems may be activated during treatment based on temperature information obtained through thermal sensors. Cooling can also be effectuated by manipulating the treatment site to increase surface area of tissues proximal to the treatment site, thereby increasing the rate of cooling of the tissues proximal to the treatment site. For example, prior to the end of delivery of energy, the patient's skin can be manipulated to establish a fold about the treatment site whereby the treatment site is disposed between two overlapping portions of the patient's skin.

In another aspect, a tissue treatment method includes delivering to a treatment site within a target tissue of a patient one or more exogenous chromophores, the exogenous chromophores having energy absorption coefficients at least two times greater than endogenous chromophores in the treatment site; and applying energy to the treatment site thereby differentially heating the target tissues containing the exogenous chromophores relative to proximal tissues not having the chromophores, wherein heat is conducted from the exogenous chromophores into the target tissues of the treatment site and the tissues are thereby remodeled. In one embodiment, the exogenous chromophores selectively absorb energy at or near the wavelength of the laser. In certain embodiments, the exogenous chromophore is a cyanine dye, such as indocyanine green, which is useful where the laser wavelength provided is in the near infrared spectra. The exogenous chromophores are delivered transdermally into the target tissues prior to application of laser energy. Heat is conducted from the exogenous chromophores to the tissues of the treatment site raising the mean temperature in the target tissues to above 40° C. Tissues proximal to the target tissues are cooled during energy delivery to a mean temperature below 47° C.

In another aspect, the invention provides a tissue treatment system. The system can include an energy source and an associated delivery assembly for selectively applying energy to be incident on the skin of a patient overlying a tissue treatment region of the patient. At least a portion of the applied energy is capable of propagating through the skin and tissue intermediate to the skin and the tissue treatment region, to the treatment region. The system also can include a temperature device adapted to generate a temperature signal representative of the temperature of at least a portion of the tissue treatment region and a controller responsive to the temperature signal to control the application of the energy to the skin whereby the temperature of the tissue treatment region is between about 40° C. and about 47° C., and the temperature of intermediate tissue proximal to the tissue treatment region is below about 40° C. Accordingly, adipocytes within the tissue treatment region are substantially damaged by the applied energy and epithelial tissue and vascular tissue proximal to the tissue treatment region are substantially undamaged by the applied energy.

The system can include one or more of the following features. The energy source can be a laser for generating the energy in the form of radiation having a wavelength in the range 800 nm to 1200 nm, for example but not limited to a 1064 nm laser. The energy source can be a laser for generating the energy in the form of radiation having an average power density of about 1-10 W/cm2, and preferably an average power density of about 4-6W/cm2. In addition, the controller can be adapted to control the applied energy to be in the form of pulsed radiation. The temperature device can include a temperature model processor for determining a model for the temperature of the treatment region, and for generating the temperature signal therefrom. The temperature device also can include a temperature sensor for detecting the temperature of at least a portion of the patient, and for generating the temperature signal therefrom. For example, the controller can be adapted to modulate the applied energy in response to the temperature signal.

The system also can include a cooling device responsive to the controller to extract heat from the treatment region. In some embodiments, the cooling device can include a heat exchanger adapted to be positioned with a heat transfer surface adjacent to the skin of the patient whereby the tissue treatment region is in thermal communication with the heat exchanger. In some embodiments, the controller controls the energy generator and the cooling device whereby the controller responsive to the temperature signal to control the application of the energy to the skin by the energy device and cooling of the treatment region, whereby the temperature of the tissue treatment region is between about 40° C. and about 47° C., and the temperature of intermediate tissue proximal to the tissue treatment region is below about 40° C. The heat exchanger can include a block of a material characterized by a relatively high thermal conductivity and a relatively high optical transmission for the energy, and the block is in relatively good thermal communication with the heat transfer surface. The block can include one or more channels passing therethrough, wherein the channels are adapted to pass a liquid heat transfer agent therethrough such that the agent is in relatively good thermal communication with the heat transfer surface. In some embodiments, the channels of the heat exchanger are substantially parallel to the heat transfer surface and/or the channels of the heat exchanger are mutually parallel.

### BRIEF DESCRIPTION OF THE FIGURES

FIG. 1 illustrates the absorption coefficients of skin chromophores and ICG solutions at concentrations of 65 and 650 micromolar.

FIG. 2 shows the temperature profile within the fat layer, using pulsed radiation to maintain a hyperthermic temperature range of the fat layer between about 42 and about 46 degrees C.

FIG. 3 illustrates a tissue fold, with radiation applied from two opposing sides of the fold. By manipulating the treatment site, the surface area of the dermal tissue is increased, while the target tissue is relatively contained by comparison. This permits greater cooling of the dermal tissue while permitting greater energy deposition in the target tissue.

FIG. 4 shows typical time/temperature profiles within abdominal adipose tissue using various power densities.

FIG. 5 shows human adipose tissue at 1-month post treatment. FIG. 5a provides a histological cross section of treated tissue showing a deep layer of necrotic adipose tissue. FIG. 5b illustrates a fat specimen from treated tissue.

### DETAILED DESCRIPTION

At the sub-cellular level, many studies have shown that the plasma membrane (containing both protein and lipid) is sensitive to external heat, and as such has been the primary target of heat-based cellular disruptive treatments. Besides the cell's plasma membrane, some other systems/organelles having similar lipid bilayer morphologies (including constitutive systems, mitochondria, ribosomes, the Golgi apparatus, lysosome, centrosome, and the endoplasmic reticulum) as well as the cytoskeleton and structural proteins are possible targets to cause cell injury and disruption. Usually, supraphysiological thermal insult is a complex matter with thermal morphological and functional alterations of multiple organelles, and always has a pleotropic (i.e., multi-target) effect on cells.

Because the lipid bilayer components of the adipocyte cell membranes are held together only by forces of hydratation, the lipid bilayer is the most vulnerable to heat damage. Even at temperatures of only 6° C. above physiological normal (i.e. about 43° C.), the structural integrity of the lipid bilayer is lost (see, Moussa N, Tell E, Cravalho E. “Time progression of hemolysis or erythrocyte populations exposed to supraphysiologic temperatures” J Biomech Eng 1979, 101:213-217). In 1989, Gaylor and Rocchio measured the stability of mammalian skeletal muscle cell membranes in isolated cell culture to supraphysiologic temperature by determining the kinetics of onset of altered membrane permeability to intracellular carboxyfluorescein dye and proposed a set of coefficients for cell membrane rupture. They found that the supraphysiologic temperatures damaged membranes at a rate which was temperature-dependent and that cell membrane lysis was probably the initial destructive event of tissue damage. The cell membranes showed evidence of damage when heated and maintained at 45° C. for more than 5 minutes (see, Gaylor, D. C. “Physical mechanism of celluar injury in electrical trauma” Massachusetts Institute of Technology. Ph. D. Dissertation. (1989).

After injury, some tissue such as the epidermis of skin can totally regenerate. Tissue regeneration is initiated by production of various growth factors. Vascular and fibroblast growth factors stimulate new blood vessel growth, fibroblast proliferation and collagen formation feed and support the functioning regenerated tissue. On the other hand, tissues such as adipose tissue only partially regenerate over a long period of time (over years).

In a typical tissue remodeling treatment, it is primarily the adipocytes underneath the skin surface, that are targeted. For a given trans-dermal laser treatment, the light has to traverse the dermis, which contains various chromophores. This reduces the energy that can be selectively deposited into deeper tissues, and it causes heating and undesirable thermal effects through the dermis and at the skin surface.

To overcome the problem of unwanted thermal effects on non-target tissues, we disclose several approaches. One approach involves application of an exogenous chromophore to a treatment site prior to delivery of trans-dermal radiation to the treatment site, the chromophore enhancing the selective energy absorption by target tissues at locations having the chromophore, i.e., within deep tissues, such as deep dermis and subdermal layers, hypodermis and superficial fascia. Another approach involves various treatment methods that all seek to control temperature of the treatment site, and include such techniques as pulsed radiation, tissue manipulation, external cooling or real-time temperature monitoring, as well as combinations of these with or without using of exogenous chromophores.

### Exogenous Chromophores

In one exemplary method, an exogenous chromophore is introduced to a treatment site prior to treatment. The chromophore is delivered through various techniques know in the art including injection, e.g., a needle syringe, a tattoo gun, or a needle-free hypodermal injection device which creates an ultra-fine stream of high-pressure fluid that penetrates the skin and delivers the chromophore into the target site.

A useful exogenous chromophore is exemplified by one of any of the available medical or food-grade dyes having a higher energy absorption at a defined wavelength (of the chosen therapeutic light source) as compared to any endogenous chromophores found within human tissues at the treatment site (such as water, hemoglobin, melanin etc.). When selecting exogenous chromophores, a higher energy absorbance differential is preferred. The particular selection depends on the subject to be treated, the natural pigmentation of the treatment site, the physiology and morphology of the treatment site, and the desired outcome of the treatment, e.g., aggressive remodeling of tissues or minor smoothing of the site. Secondary considerations include the susceptibility of the exogenous chromophore to photodamage and the ability of the body to clear excess chromophore from the treatment site. Persistence of visible quantities of exogenous chromophore at the treatment site following treatment is undesirable.

The laser is selected from one of any of a number of currently available sources. An appropriate laser is one whose penetration depth is comparable to or longer than the depth of dermal tissues at the thickest point within the treatment area. The wavelength of operation for lasers meeting this requirement is variable as well, but currently preferred systems employ wavelengths in the visible or near infrared regions of the electromagnetic spectrum, and more preferably in the near infrared spectrum. One example of preferable wavelength is 800 nm. This wavelength has minimum absorption in blood and water which are major endogenous chromophores in human skin. By way of further nonlimiting example, in the case where an 800 nm wavelength laser source is chosen as the energy source, any chromophores with high absorption near 800 nm are good initial choices. Indocyanine Green (ICG) is one possible choice for an exogenous chromophore, due to its absorption character but also its commercial accessibility and proven record of safety for human use. It is a cyanine dye and has been used widely in medical diagnostics for determining cardiac output, hepatic function, and liver blood flow, and for ophthalmic angiography. It has a peak spectral absorption at about 800 nm.

An embodiment that allows the procedure above includes an energy source such as a laser, a trans-dermal injection system which could deliver the chosen chromophore into fat layer to enhance the light absorption of fat, optionally a surface cooling system such as a chiller, and possibly thermal sensors in the device or imaging systems in the surgical theater, to monitor the treatment parameters, such as tissue temperature in deep tissue and on skin surface, etc. The laser can be one of any of a number of available sources whose penetration depth is deeper than the thickness of skin at treatment area. The preferred wavelength of operation of lasers suitable for the above procedure depends in part on the absorption profile of the exogenous chromophore if one is used, but currently preferred wavelengths are in the visible or near infrared regions of the electromagnetic spectrum, more preferable in the near infrared regions. One example of a currently preferred wavelength is 800 nm. This wavelength has deeper penetration depth than human skin thickness. In order to enhance the absorption of light in fat layer, a trans-dermal injection of one or more selected exogenous chromophores is an option.

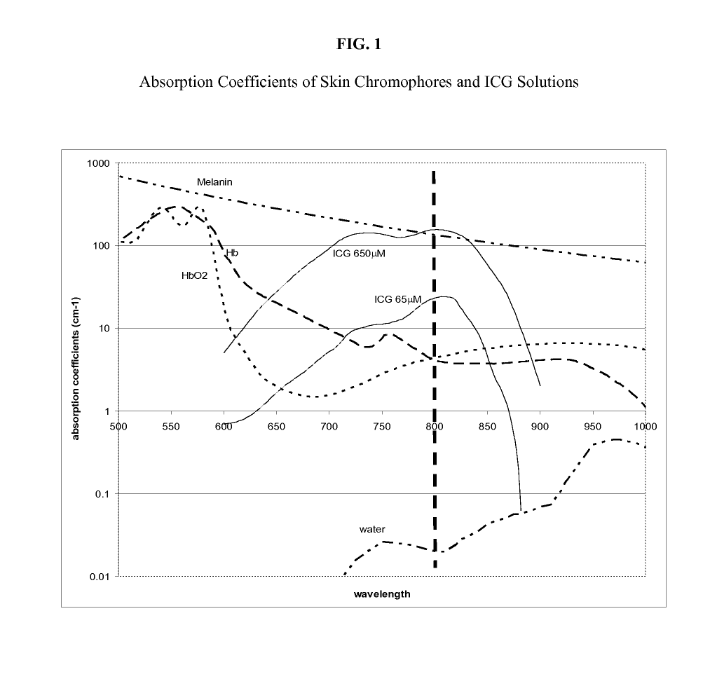

FIG. 1 compares the absorption coefficients of 65 micromolar and 650 micromolar ICG solutions, to absorption coefficients of some major endogenous chromophores found naturally in human dermis. At 800 nm, a 650 micromolar ICG solution has 14 times higher energy absorption than blood (for both hemoglobin and deoxyhemoglobin), and its energy absorption is more than 7700 times higher than water. Although human melanin has comparable absorption coefficient, it primarily locates in skin epidermis within the first 100 micrometers of dermal tissue. This endogenous chromophore does cause some heating of the dermis in the treatment beam path with consequent potential for thermal damage to tissues within or proximal to that path, but this effect could be protected against by sufficient external surface cooling of the skin if necessary. Furthermore, it is less of a concern for lighter pigmented skin due to its lower volume density in lighter skin types. The volume fraction (fv) of melanosomes in epidermis varies with skin color: for light skinned Caucasians, fv=1-3%; for well-tanned Caucasians and those of Mediterranean lineage, fv=11-16%; and for persons of African decent the variability is much higher, where fv=18-43%.

### Thermal Control

Adaptations to limit thermal damage to non-target tissues are used with the above exogenous chromophores or can be used themselves. Equipment such as thermal sensors, imaging systems and laser control systems that monitor the treatment parameters, e.g., position of the laser, contact of cooling plate with treatment surface, duration and dosage of laser energy at the treatment site, temperature of the target site within deep tissues and on the skin surface are described in our U.S. patent application Ser. No. 12/135,967 incorporated herein by reference. Contact cooling systems for surgical application are similarly known in the art, and are useful in combination with the approaches described herein. These all provide methods for controlling the deposition of thermal energy in both the target tissues and the non-target tissues within the treatment zone. For example, periodic pulsing of the laser provides another means of modulating heat deposition in the treatment site, as described in our application PCT US2010/026211 incorporated herein by reference.

The hyperthermic treatment of fatty tissue, which at a treatment site raises the mean tissue temperature above about 40° C., and more preferably about 42-46° C. induces thermal injury to adipocytes in the treatment area. Notably, 46° C. is not the upper limit of treatment, as higher temperatures (47-50° C. or more e.g. 60° C., 70° C., 80° C., etc) denatures cells and even ablate tissues, but these also raise the mean heat level in the non-target tissues causing collateral damage. Such heat-induced injury triggers the adipocytes to undergo apoptosis or lipolysis. The residual cellular debris is gradually removed by the body through inflammation and the resultant immune system clearing process, which takes weeks to months depending on the extent of injury at the site. Since the regeneration process of adipose tissue is very slow (over years), the total volume of fat within the treatment area decreases due to loss of adipocytes that would otherwise act as storage units for such fat.

To accomplish this, laser irradiation of the treatment site is conducted in order to achieve a supraphysiological temperature (greater than 37° C.) in the treatment site over a period of time—for example, a few minutes to hours or so depending on the particular temperature applied. Various preferred embodiments endeavor to confine substantially, the hyperthermic region to fat layers in the target tissue, while keeping dermal temperatures in the treatment are below injury threshold (i.e., lower than about 46-47° C.). By choosing the laser parameters (such as radiation pattern, fluence and exposure time, etc) and factoring the cooling rate on the skin surface, an optimized temperature profile/gradient in the target tissue is achieved.

One technique, Selective Photothermolysis (SPTL) has been widely used for many photothermal therapies, such as hair removal and superficial vascular treatment. The objective behind SPTL is to choose an energy source, e.g., laser light, having a specific wavelength that is selectively or preferentially absorbed by the targeted tissue (such as adipocytes and lipid bilayer structures), with less absorption and therefore less thermal effect on the surrounding tissues (such as epidermis). Optimal SPTL is achieved when the targeted tissue has a much higher energy absorption compared to other surrounding tissues. Frequently, this effect is controlled by selecting lasers having particular wavelengths for specific cosmetic purposes. But in certain procedures, selection of wavelength alone is not itself sufficient to create a large enough energy absorption differential between target and non-target tissues to achieve optimal therapeutic effects without some degree of damage to surrounding non-target tissues. We have developed several approaches which increase the energy absorption differential and control heating at the treatment site, in order to minimize collateral damage of non-target tissues. Each will be discussed in turn.

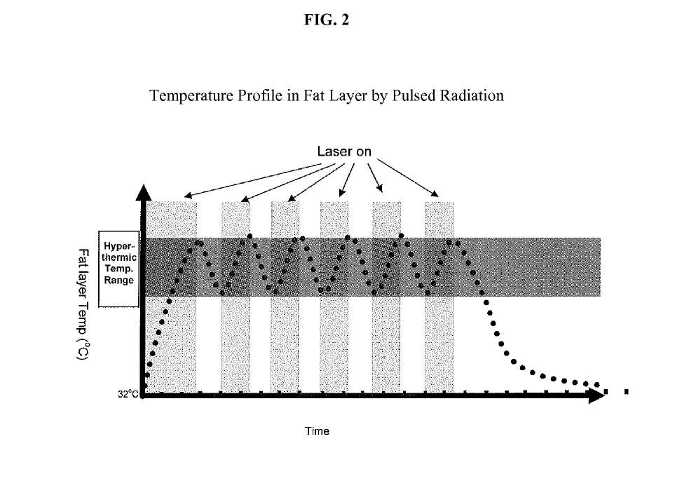

One method of controlling temperature at the treatment site involves modulating the radiation exposure through pulsed applications of laser light. As shown in FIG. 2, a near infrared laser having a wavelength of 1064 nm is selected based on its tissue penetrance and relatively low absorption by melanin and water, the major chromophores in the skin. Exemplary power densities are 1-10W/cm2, and a particularly useful range is about 4-6W/cm2. To maintain an appropriate hyperthermic temperature range in the target tissue (about 40-45° C. in the fat layer) while avoiding pain and other unwanted side effects related to overheating, the laser is pulsed, generating an on/off pattern, which causes the temperature to cycle within the appropriate hyperthermic temperature range. With the laser on, the temperature rises to the upper limits of the desired range. A periodic pause permits temperatures in the target site to drop, and optionally the cooling can be further enhanced by using external devices. Laser radiation resumes before tissue temperature drops below the appropriate hyperthermic temperature range. The pulses are repeated for the duration of the treatment (e.g., about 16 minutes as illustrated).

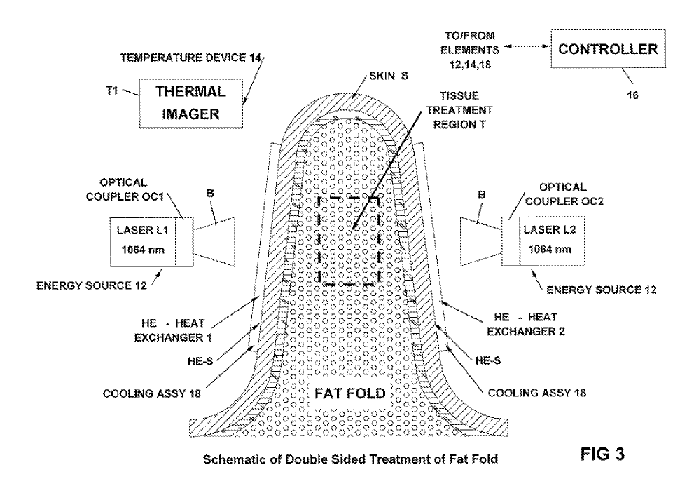

FIG. 3 illustrates one embodiment, where a patient's tissue is physically manipulated to create a tissue “fold” bounded by the patient's skin S and having an internal central region of subcutaneous adipose tissue. T, the “treatment region”. A tissue treatment system 10 is positioned to selectively apply energy to the patient's skin S at regions overlying the treatment region T. The energy provided is capable of propagating through the skin S and tissue intermediate to the skin and the tissue treatment region, to the treatment region T.

The tissue treatment system 10 includes an energy source and an associated delivery assembly 12, a controller 16, a cooling assembly 18 and optionally, a temperature device 14. In the illustrated embodiment of FIG. 3, the energy source includes A pair of lasers L1 and L1, each with an associated delivery assembly, in the form of beam-forming optical couplers OC1 and OC2 respectively. In other embodiments, a different form and number of energy sources can be used.

The illustrated optional temperature device 14 is in the form of a thermal imager TI, which generates a temperature signal representative of the patient's tissue based on the thermal footprint of the skin S near the treatment tissue. Other forms of generating a temperature signal are used in other embodiments, including a processor which generates estimates of the temperature of the treatment tissue and adjacent tissue, based on a thermal model of the patient and the energy applied to and extracted from the treatment tissue, directly or indirectly.

The cooling assembly 18 is in the form of a cooler having a heat exchanger HE having a surface HE-S adapted for intimate thermal contact with a portion of the patient's skin S which, in turn, is in thermal communication with the tissue treatment region T. In various embodiments, the heat exchanger may be adapted to extract heat across the patient's skin by a liquid heat transfer agent passing therethrough, by a thermoelectric heat transfer device or another known form of controlled cooling device. In one form, using a liquid cooling agent, the cooling agent flows through tubes in a structure which is transparent to the laser radiation, so that the cooling structure can be placed directly against the patient's skin, overlying the tissue treatment region. The temperature and flow rate of the cooling agent can be adjustably controlled by the controller, to maintain the temperature of the patent's tissue in the tissue treatment region in the desired range. In addition, the heat exchanger can be rigid or semi-rigid, and the heat exchanger can be flexible, for example, permitting the heat exchanger to conform to the skin surface.

The energy source and associated delivery assembly 12, the temperature device 14 (and its generated temperature signal) and the cooling assembly 18, are all coupled to the controller 16. Those elements operate under the control of controller 16. to control the application of the energy via beams B to (and optionally extraction of energy across surfaces HE-S from) the skin of the patient whereby
i. the temperature of the tissue treatment region is between about 40° C. and about 47° C., and
ii. the temperature of intermediate tissue proximal to the tissue treatment region is below about 40° C.,
   whereby adipocytes within the tissue treatment region are substantially damaged by the applied energy and epithelial tissue and vascular tissue proximal to the tissue treatment region are substantially undamaged by the applied energy.

In operation, the skin fold of the patient is irradiated via laser beams B (and also cooled) from opposing external sides. The convergence/overlap of radiation along the light paths increases the heat flux into the tissue fold, but the dermal cooling occurring at each side of the fold behaves similar to single beam approaches. This enhances the efficacy of adipose tissue heating leading to better fat reduction, while decreasing undesired treatment site tissue damage. In other applications of the tissue treatment system, operation may be similarly performed, but without manipulating the patient's skin to form a fold, thereby attaining radiation from just a single side of the tissue treatment region.

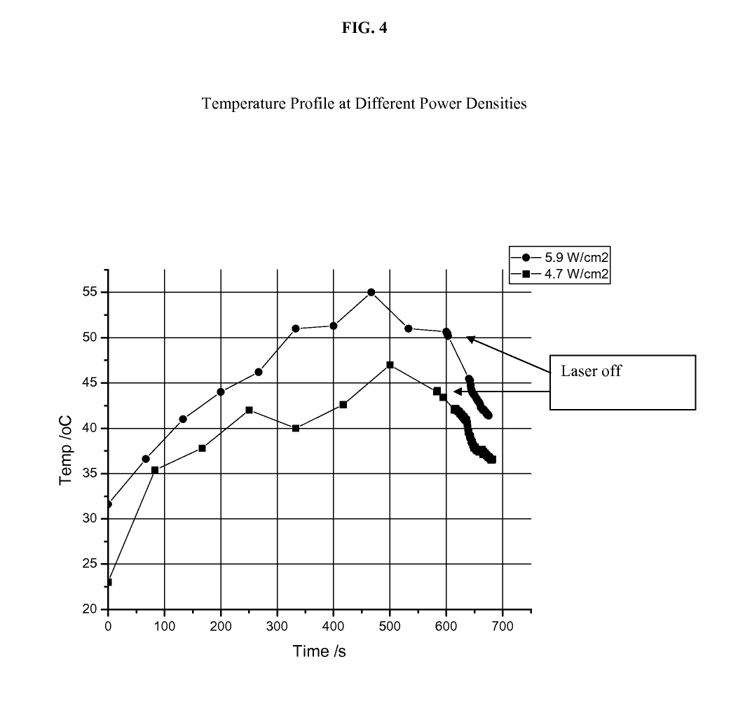

FIG. 4 shows the time/temperature profiles in vivo, for human abdominal fat treated using a 1064 nm wavelength laser with an 18mm spot size, using the double sided treatment configuration shown in FIG. 3 above. Two power densities were used, 4.7 and 5.9W/cm2. External air cooling of the site was employed to maintain a skin surface temperature of below 30° C., as monitored by an external thermal camera. Temperature in the subcutaneous fat layer was monitored by a thermal probe inserted about 1 cm below the skin, the position reflecting the position at which Tmax was observed. Temperatures exceeded 40° C. after 133 seconds (at 5.9W/cm2) or 250 seconds (at 4.7W/cm2) respectively.

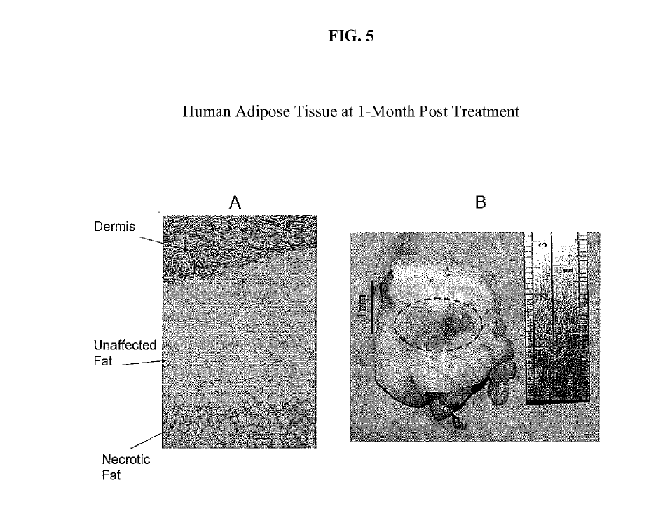

FIG. 5 illustrates the effect on human abdominal tissue at 1 month post-treatment. A 1064 nm laser having an 18 mm spot size and employing a power density of 5.1W/cm2 was used for the 30 minute treatment, pulsed such that the laser was “on” for about 66% of the treatment time. FIG. 5a shows a tissue biopsy stained with H&E, that reveals a necrotic region deep in the adipose tissue below the dermal layer. FIG. 5b illustrates the gross morphology of the fat specimen in cross section. A necrotic zone is seen in the middle portion of the tissue, shown within the superimposed oval. In both tissue samples, the dermal tissues were not damaged.

### Equivalents

Other variations on the invention are possible, and deemed equivalent to and within the scope of the invention described. For example, while uniform beam laser systems have been described above, a non-uniform beam can be employed. Such non-uniform output beams are described in our U.S. Pat. No. 7,856,985 and application PCT/US 10/26432, both incorporated herein by reference. Another equivalent source of deep energy delivery is a focused ultrasound device having a focal depth longer than the skin thickness at the treatment location. In another embodiment, a focused ultrasound device having a scanning system is employed, which can overlay the focused ultrasound energy uniformly over the whole treatment area. In still other embodiments, RF energy is used to generate the hyperthermic condition in the target tissue. Other modifications to the present system and methods will become apparent to those having skill in the relevant medical arts in view of the teachings contained herein.

We claim:

1. A tissue treatment method comprising:
delivering to a treatment site within a tissue of a patient sufficient energy to heat the tissue to a mean temperature above 40° C.; and
maintaining a temperature below 47° C. within and proximal to the treatment site, thereby damaging adipocytes within the treatment site without substantial damage to epithelial or vascular tissues proximal to the treatment site.

2. The method of claim 1, wherein the heating of tissues within the treatment site is accomplished with laser radiation having a wavelength ranging from 800 nm to 1200 nm.

3. The method of claim 1, wherein the heating of tissues within the treatment site is accomplished with laser radiation having a wavelength of 1064 nm.

4. The method of claim 1, wherein the heating of tissues within the treatment site is accomplished with laser radiation having an average power density of about 1-10W/cm2.

5. The method of claim 1, wherein the heating of tissues within the treatment site is accomplished with laser radiation having an average power density of about 4-6W/cm2.

6. The method of claim 1, wherein energy is delivered to the treatment site in the form of periodic pulsed radiation.

7. The method of claim 1 wherein the step of maintaining a temperature below 47° C. within and proximal to the treatment site is effected at least in part by determining the temperature as a function of time of the treatment site, and modulating the delivery of energy from the energy source in response thereto.

8. The method of claim 7, wherein the step of determining the temperature is effected by thermal imaging sensors.

9. The method of claim 7 wherein the step of maintaining a temperature below 47° C. within and proximal to the treatment site is effected at least in part by modulating the delivery of energy from the energy source.

10. The method of claim 1, wherein the heating of tissues within the treatment site occurs for about 2 to about 60 minutes.

11. The method of claim 10, wherein the heating of tissues in the treatment site further comprises simultaneous cooling of tissues at the treatment site.

12. The method of claim 11, wherein cooling is intermittent during energy delivery.

13. The method of claim 11, further comprising the step of:
prior to the end of delivery of energy, manipulating patient's skin to establish a fold about the treatment site whereby the treatment site is disposed between two overlapping portions of the patient's skin.

14. A tissue treatment method comprising:
delivering to a treatment site within a target tissue of a patient one or more exogenous chomophores, the exogenous chromophores having energy absorption coefficients at least two times greater than endogenous chromophores in the treatment site; and
applying energy to the treatment site thereby differentially heating the target tissues containing the exogenous chromophores relative to proximal tissues not having the chromophores, wherein heat is conducted from the exogenous chromophores into the target tissues of the treatment site and the tissues are thereby remodeled.

15. The method of claim 14, wherein the energy is provided using a laser.

16. The method of claim 15, wherein the exogenous chromphores selectively absorb energy at or near the wavelength of the laser.

17. The method of claim 16, where one of the exogenous chromophores is a cyanine dye.

18. The method of claim 17, wherein one of the exogenous chromophores is indocyanine green and the laser wavelength provided is in the near infrared spectra.

19. The method of claim 14, wherein the one or more exogenous chomophores are delivered transdermally into the target tissues prior to application of energy.

20. The method of claim 14, wherein heat is conducted from the exogenous chromophores into the target tissues of the treatment site raising the mean temperature in the target tissues to above 40° C.

21. The method of claim 14, wherein tissues proximal to the target tissues are cooled during energy delivery.

22. A tissue treatment system comprising:
A. an energy source and an associated delivery assembly for selectively applying energy to be incident on the skin of a patient overlying a tissue treatment region of the patient, wherein the at least a portion of the applied energy is capable of propagating through the skin and tissue intermediate to the skin and the tissue treatment region, to the treatment region,
B. a temperature device adapted to generate a temperature signal representative of the temperature of at least a portion of the tissue treatment region,
C. a controller responsive to the temperature signal to control the application of the energy to the skin whereby
i. the temperature of the tissue treatment region is between about 40° C. and about 47° C., and
ii. the temperature of intermediate tissue proximal to the tissue treatment region is below about 40° C.,
whereby adipocytes within the tissue treatment region are substantially damaged by the applied energy and epithelial tissue and vascular tissue proximal to the tissue treatment region are substantially undamaged by the applied energy.

23. The system of claim 22, wherein the energy source is a laser for generating the energy in the form of radiation having a wavelength in the range 800 nm to 1200 nm.

24. The system of claim 22, wherein the energy source is a laser for generating the energy in the form of radiation having a wavelength of substantially 1064 nm.

25. The system of claim 22, wherein the energy source is a laser for generating the energy in the form of radiation having an average power density of about 1-10 W/cm2.

26. The system of claim 22, wherein the energy source is a laser for generating the energy in the form of radiation having an average power density of about 4-6W/cm2.

27. The system of claim 22, wherein the controller is adapted to control the applied energy to be in the form of pulsed radiation.

28. The system of claim 22, wherein the temperature device includes a temperature model processor for determining a model for the temperature of the treatment region, and for generating the temperature signal therefrom.

29. The system of claim 22, wherein the temperature device includes a temperature sensor for detecting the temperature of at least a portion of the patient, and for generating the temperature signal therefrom.

30. The system of claim 29, wherein the controller is adapted to modulate the applied energy in response to the temperature signal.

31. The system of claim 29, further comprising:
D. a cooling device responsive to the controller to extract heat from the treatment region.

32. The system of claim 31, wherein the cooling device includes a heat exchanger adapted to be positioned with a heat transfer surface adjacent to the skin of the patient whereby the tissue treatment region is in thermal communication with the heat exchanger.

33. The system of claim 32, wherein the controller controls the energy generator and the cooling device whereby the controller responsive to the temperature signal to control the application of the energy to the skin by the energy device and cooling of the treatment region whereby
i. the temperature of the tissue treatment region is between about 40° C. and about 47° C., and
ii. the temperature of intermediate tissue proximal to the tissue treatment region is below about 40° C.

34. The system of claim 31, wherein the controller controls the energy generator and the cooling device whereby the controller responsive to the temperature signal to control the application of the energy to the skin by the energy device and cooling of the treatment region whereby
i. the temperature of the tissue treatment region is between about 40° C. and about 47° C., and
ii. the temperature of intermediate tissue proximal to the tissue treatment region is below about 40° C.

35. The system of claim 32, wherein the heat exchanger includes a block of a material, wherein:
i. the material is characterized by relatively high thermal conductivity,
ii. the material is characterized by a relatively high optical transmission for the energy,
iii. the block is in relatively good thermal communication with the heat transfer surface, and
iv. the block includes one or more channels passing therethrough, wherein the channels are adapted to pass a liquid heat transfer agent therethrough whereby the agent is in relatively good thermal communication with the heat transfer surface.

36. The system of claim 35, wherein the channels of the heat exchanger are substantially parallel to the heat transfer surface.

37. The system of claim 36, wherein the channels of the heat exchanger are mutually parallel.

38. The system of claim 35, wherein the channels of the heat exchanger are mutually parallel.
